(19) United States
(12) Patent Application Publication
(10) Pub. No.: US 2025/0367361 A1
(43) Pub. Date: Dec. 4, 2025

(54) DEVICES, SYSTEMS AND METHODS FOR PROTECTING THE BRAIN IN CRANIECTOMY PATIENTS

(71) Applicants: MOR RESEARCH APPLICATIONS LTD., Ramat Gan (IL); SHAARE ZEDEK SCIENTIFIC LTD., Jerusalem (IL)

(72) Inventors: Yaron SACHER, Ramat Gan (IL); Lihi MANSANO, Ramat Gan (IL); Gustavo Gabriel RAJZ, Jerusalem (IL); Revital PERY-SHECHTER, Rishon Letzion (IL)

(21) Appl. No.: 18/878,042

(22) PCT Filed: Jun. 22, 2023
(86) PCT No.: PCT/IB2023/056474

§ 371 Date: Dec. 22, 2024

(30) Foreign Application Priority Data
Jun. 22, 2022 (IL) 294216

(51) Int. Cl. A61M 1/00; A61F 13/00; A61F 13/05; A61F 13/12

(52) U.S. Cl. CPC A61M 1/962; A61F 13/00987; A61F 13/05; A61F 13/12; A61M 2205/3317; A61M 2205/3327; A61M 2205/3334; A61M 2210/0693; A61M 2230/62

(57) ABSTRACT

Embodiments pertain to a cranial cap, adapted for covering, at least partially or fully, a skin flap of a patient's head having undergone a craniectomy, the cranial cap comprising: a cap body; a flexible sheet adaptable to adhere to the head; wherein the cap body and the flexible sheet are sealed to form a fluid-tight cavity; a tubing connector port for removably bringing a pump in fluid communication with the cavity such to enable controlling fluid pressure in the cavity; and at least one sensor adapted and disposed to sense at least one characteristic relating to a skin flap shape for responsively providing an output descriptive of or relating to the skin flap shape; and patient posture and/or patient movement.

### CROSS-REFERENCE TO RELATED APPLICATIONS

This patent application claims the benefit and priority from IL patent application 294216, filed Jun. 22, 2022, and which is incorporated herein by reference in its entirety.

### BACKGROUND

Traumatic brain injury (TBI) such as a severe fall, car accident or gunshot wound can compromise the skull, and cause intracranial hypertension. Intracranial hypertension may necessitate an intervention and removal of a portion of the skull (Craniectomy) to allow a swelling brain to expand without being squeezed in an effort to prevent tissue damage with a potential compromise of cerebral circulation and function, and to treat edema. Craniectomy may sometimes also be performed on patients that suffered of a stroke, as well of intracranial bleeding.

Cranioplasty, or surgery to replace the removed bone flap is not to be performed until the swelling subsides, which can take several weeks, usually at least two months.

### BRIEF DESCRIPTION OF THE DRAWINGS

The figures illustrate generally, by way of example, but not by way of limitation, various embodiments discussed in the present document.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The number of elements shown in the Figures should by no means be construed as limiting and is for illustrative purposes only. The figures are listed below.

FIG. 1 schematically depicts a craniectomy region where a cranial bone portion is removed leaving an aperture in the remaining cranial bone which is covered over by a skin flap.

FIG. 2 schematically depicts the “sinking skin syndrome” which is the result of decreased intracranial pressure Pcran applied onto the inner skin surface of the skin flap relative to atmospheric pressure Patm exerted onto the external skin surface of the skin flap.

FIG. 3A schematically depicts a cranial cap engaging with a patient's head, the cranial cap adapted for covering, at least partially or fully, a skin flap of a patient's head having undergone a craniectomy, prior to the application of suction to lift the sunken skin, according to some embodiments.

FIG. 3B schematically depicts the cranial cap of FIG. 3A and lifted skin following the application of suction, according to some embodiments.

FIG. 4 schematically depicts the cranial cap apart from the head of patient, according to some embodiments.

FIG. 5A schematically depicts a cranial cap operably engaged with a patient's head, according to some embodiments.

FIG. 5B schematically depicts a cranial cap operably engaged with a patient's head, according to some other embodiments.

FIG. 6A schematically depicts a system for preventing Syndrome of the Trephined (SoT), according to some embodiments.

FIG. 6B schematically depicts an underside of a cranial cap, according to some embodiments.

FIG. 7 schematically depicts a coupling member for coupling a cranial cap to a patient's head, according to some embodiments.

FIG. 8 schematically shows a flowchart of a method for lifting sunken skin of a craniectomy region, according to some embodiments.

### DETAILED DESCRIPTION

Aspects of the present invention pertain to patients who have undergone a craniectomy but have not yet undergone a cranioplasty. As a result, there is no bone between the skin flap and the brain tissue. There is a known medical syndrome referred to as Syndrome of the Trephined (SoT), also known as the “Sunken brain and Scalp Flap Syndrome”, “syndrome of the sinking skin flap” (SSSF) or “motor trephined syndrome” (MTS), in which neurological deterioration occurs following removal of a large skull bone flap, due to excess depressurization of the intracranial pressure. In some cases, SoT may be related to posture, and/or to transition between postures, and/or a rate of transition between postures. Symptoms of excess intracranial depressurization include, for example, motor deficits, cognitive deficits, language deficits, and headaches. SoT was documented in patients after craniectomy, as well as its resolution after cranioplasty. Hence, while it is imperative to prevent hypertension, it may also be important to prevent or counteract, at least to some extent, intracranial depressurization.

Embodiments of the present invention pertain to devices, systems and methods configured to reduce the extent or prevent the occurrence of SoT entirely, for example, by controllably raising the skin flap, e.g., by controlling the pressure applied onto the skin flap, for example, to offset for the influence of the atmospheric pressure to which the skin flap is subjected to.

Accordingly, embodiments of the devices, systems and methods disclosed herein are configured to counteract excess flap depression, counteract progress of flap depression, to counteract the formation of SOT, to remedy existing SOT, to slow down or reverse the progress of sinking skin, and/or the like.

In some embodiments, the device and/or system may be configured to create a cavity that can be (e.g., continuously) and non-invasively monitored to correct for (e.g., excess) pressure imbalance between the intracranial compartment and the outer atmospheric pressure by applying negative pressure over the skin flap. In some examples, relative negative pressure may be generated in the cavity in a continuous manner, in an intermittent (e.g., periodic) manner, in a dynamic manner, and/or in an adaptive manner.

The system may be configured to provide an output indicative regarding such pressure imbalance. In some examples, the output may represent an alert that is provided to a user of the system. The system user may be, for example, a medical professional, the patient, or both. In some examples, the output may be provided to a controller of the system for controlling the operation of an actuator (e.g., pump device) of the system, as outlined herein in more detail. The actuator may for example be any one of the following, a pump, such as, for example, a dynamic pump (e.g., centrifugal pump), and a displacement pump (e.g., piston pump, a diaphragm pump); and/or electroactive (e.g., polymeric) material.

In some embodiments, the system is configured to monitor and control the extent of SoT or prevent the occurrence of SoT by additionally taking into consideration patient movement and/or posture. For example, the devices, systems, and methods may employ at least one inertial and/or non-inertial sensor for monitoring flap height, cap deformation, patient movement and/or posture.

In some examples, the sensors may comprise an electroactive (e.g., polymer) material that outputs an electric signal responsive to material deformation, e.g., indicative of flap depression. Optionally, a measured magnitude in material deformation may be translated into a shear stress and/or normal stress measurement. Based on outputs provided by these sensors to a controller (e.g., based on a voltage output or change voltage output by electroactive material in response to tissue deformation), flap or SOT-related characteristics may be monitored by outputs provided by these sensors.

In some embodiments, the cap may comprise actuators for causing the actuation of various flap raising functionalities for example, by changing mechanical characteristic of the cap such as, for example, stiffness, elasticity, flexibility and/or shape of cap, for example, upon receipt of a control signal that may be output by a controller.

The controller signal, for instance, may cause the pump to apply suction and/or the electroactive material to reshape, stiffen, relax and/or otherwise selectively and locally change mechanical characteristics (e.g., stiffness, elasticity and/or flexibility), for example, to counteract excess flap depression, counteract progress of flap depression, to counteract the formation of SOT, to remedy existing SOT, to slow down or reverse the progress of sinking skin, and/or the like.

Accordingly, the electroactive material may in some embodiments function as a (e.g., wearable) sensor, as an actuator, or both. Optionally, a first portion of electroactive material function functions as a sensor, and another portion functions as an actuator. Optionally, the same portions of electroactive material function as a sensor and as an actuator. In some embodiments, one or more portions of the cap may have different or identical electroactive material properties. In some embodiments, actuators in the form of one or more pumps and electroactive material may be employed concurrently. In some embodiments, the electroactive material may constitute part of a pump employed for aspirating fluid to reduce pressure in the cavity to counteract the formation of SOT or to cancel SOT. In some embodiments, electroactive material may function as a sensor or sensors providing outputs to circuitry and/or a controller for activating one or more pumps to counteract the formation of SOT, to remedy existing SOT, to slow down or reverse the progress of sinking skin.

In some embodiments, the devices, systems and methods may be configured to monitor one or more characteristics relating to SoT, patient postures, movements, and/or patient physical activity state (e.g., resting state vs non-resting state) for controlling and reducing, based on the one or more monitored characteristics, skin flap depression, and/or for maintaining a skin flap height within a certain range.

In some examples, the devices, systems and methods may be configured to identify a posture and/or distinguish between different patient postures, identify a change in posture, determine a rate in the change between two different postures, detect a change in patient movement, detect a direction in the patient's movement, and/or the like, and, optionally concurrently, monitor a flap contour.

A flap contour that appears relatively sunken may suggest a reduction in intracranial pressure.

Nevertheless, the patient's intracranial pressure can also be affected by their posture and/or movement; and/or change of rate of the posture and/or movement, which can cause corresponding (e.g., excess) alterations in the flap contour.

For example, the flap contour of an upright standing patient may be increasingly sunken, compared to the flap contour of a supine patient. Therefore, lying down may reduce a flap depression, while standing up may cause flap depression or exacerbate an existing flap depression.

Therefore, disregarding patient position and/or movement may result in that flap depressions are erroneously identified as a clinically undesired flap or excess flap depression.

It is thus beneficial to regulate, counteract and/or observe flap depression not solely based on its physical characteristics, but also considering the patient's posture and/or movements. Considering the patient's postures and/or movements allows them to engage in their daily activities without the need to maintain a specific posture solely for the purpose of effective flap contour monitoring by the system.

Accordingly, in some examples, different flap- or skin depression-thresholds for initiating rising the skin flap may be defined depending on the patient's postures, movements, transition rate between posture, movement rate, and/or the like. Such depression threshold may pertain to the magnitude of the flap depression relative to a nominal (e.g., desired) flap depression that is within a desired range, e.g., is within a range of values that are defined as “non-depression”.

For example, the system may select or employ a supine-depression threshold in the event the patient is in supine position. The supine-depression threshold may be of lower magnitude compared to an upright depression threshold that may be used or selected when the patient is in upright or standing position. In such an example, a depression that exceeds the supine-depression threshold may be shallower than a depression that exceeds that upright-depression threshold.

In another example, the supine-depression threshold may be of higher magnitude compared to an upright depression threshold that may be used or selected as a threshold in cases where the patient is in upright or standing position. In such an example, a depression that exceeds the supine-depression threshold may be steeper than a depression that exceeds that upright-depression threshold.

Based on sensor output provided in supine position, an alert may be output warning of excess depression, and/or a controller may start causing aspiration from the cavity to cause rising of the depressed skin flap if, for example, a sensed depression parameter value exceeds the supine depression threshold. Analogously, based on sensor output produced when the patient is in upright position, an alert may be output warning about excess depression, and/or the controller may start causing aspiration of fluid from the cavity to cause rising of the (e.g., depressed) flap if, for example, a monitored depression value exceeds the upright depression threshold.

Additional and non-limiting patient postures and/or transitions therebetween that may be taken into consideration during monitoring and for controlling the patient's skin flap depression include the patient's right and/or left lying posture.

In addition to monitoring patient postures and extent of flap depression for providing a corresponding alert and/or initiating corrective measures to decrease excess flap depression, for example, by applying suction, and/or electrical energy for changing characteristics or of electroactive material of the cap, embodiments may also take into consideration bone flap and/or craniectomy location, e.g., receive information about craniectomy classification, including, for example, frontal, temporal, parietal and/or occipital craniectomy.

In some embodiments, depending on a rate of transition between patient two or more different patient postures, transition frequency between two or more different patient postures, the sequence between two different postures, and/or the like, an alert and/or control of flap-rising suction may or may not be employed.

Additional or alternative depression-related parameter values or characteristics that may be taken into account for monitoring and, optionally, controlling skin flap depression, include the skin flap sinking and/or rising rate. For example, if the skin flap sinking rate exceeds a sinking threshold rate, the controller may be employed to raise the skin flap and/or to counteract skin flap depression, optionally even if the magnitude of the depression does not yet exceed a depression threshold.

In some embodiments, the system is configured to form a pressure-controllable cavity which is configured to allow controllably reduce pressure in the formed cavity for preventing unwanted skin flap deformation (e.g., excess depression), eliminating unwanted skin flap deformation, counteract and/or reducing unwanted skin deformation (e.g., excess depression). This way, the continuity of the rehabilitation process may be improved or ensured.

The cavity may be a fluid-tight (also: substantially fluid-tight) cavity. In some examples, reduction in the cavity pressure (also: creation of sub-pressure), causes lifting of the skin flap away from the patient's brain tissue, thereby correcting for, or preventing undesired skin flap deformation. It is noted that term “fluid” as used herein may encompass gas, liquid, and/or air.

The direction in which the skin flap is moved away from the patient's brain tissue may herein be referred to as “distal direction”. Correspondingly, a proximal direction indicates a direction in which the skin flap “sinks” onto the brain tissue or forms a depression, due to intracranial depressurization.

In some embodiments, the pressure-controllable cavity may for example be created by a cranial cap of the system covering the skin flap. A pressure control apparatus of the system comprising a pump may be configured to control the pressure in the cavity, based on the one or more sensed skin flap shape parameters.

A skin flap shape parameter value may pertain to, for example, cavity pressure; a skin flap height, fa depression, flap height, e.g., relative to a reference value pertaining to a desired shape, and/or the like, (e.g., non-invasively) sensed by one or more sensors employed by the system.

For example, the cranial cap may comprise a pressure sensor configured to sense the pressure in the cavity. In a further example, a distance and/or proximity sensor may be employed for sensing skin flap height. Additional sensing modalities that may be employed for gathering data relating to intracranial pressure may include transcranial doppler (TCD) measurement which is based on measuring middle cerebral arterial (MCA) velocity, optic nerve sheath diameter measurement, electroactive material sensing modality, and/or the like. Further parameter values that may relate to intracranial pressure pertain to sweating, where excess sweating may be indicative of increased intracranial pressure.

Example intracranial pressure sensing modalities are outlined in ““Intracranial Pressure Monitoring—Review and Avenues for Development”, by Maya Harary et al., 5 Feb. 2018”, which is incorporated herein by reference in its entirety.

In some embodiments, the one or more sensors may be employed for sensing cap displacement relative to the patient's skull with respect to a desired reference position. In the event of sensed displacement relative to the desired reference position exceeding a displacement-related threshold value, a corresponding output may be provided.

In some embodiments, the one or more sensors may include an interface coupling (e.g., adherence) strength sensor. In the event a sensed coupling strength of the cap surface with the patient's skin surface drops below a certain coupling-strength threshold value, a corresponding output may be provided to the user.

In some embodiments, the one or more sensors may be configured to sense fluid leakage from within the cavity, and provide an output indicative of leakage of fluid.

In some embodiments, the one or more sensors may be calibrated once the cap is operably engaged with the patient's head at a desired position.

In some embodiments, at least some portions of the cap may be made of transparent material to allow substantially unobstructed view of the patient's skin flap for inspection thereof without requiring cap disengagement. In some examples, the transparent material may be covered with a selectively removable cloth to allow for visual inspection of the patient's skin flap without requiring cap disengagement.

The one or more sensors may for example be arranged on the underside of the cap body, herein referred to as “proximal” to the patient's head, and/or on the external surface of the cap body, herein referred to as “distal” from the patient body.

In some embodiments, a sensing frequency can be preset or predetermined. In some examples, the sensing may be performed continuously. In some examples, sensing a physical characteristic may be triggered based on the sensing of a physical characteristic of another sensor. For example, a first type of sensing modalities may be employed when the patient is in motion, e.g., as sensed by an accelerometer, and a second type of sensing modalities may be employed when sensor outputs are indicative that the patient is in a rest state.

In some embodiments, a level of correspondence between sensor outputs may be determined for detecting one or more of the following: false-negative outputs, false-positive outputs, sensor defects, and/or the like. The system may provide an output or alert if the level of correspondence does not meet a correspondence criterion (e.g., correlation value drops below a correlation threshold.

Aspects of embodiments pertain to a helmet, patch, cap, or similar, that can be worn by the patient, optionally configured or formed taking into consideration the (e.g., type of) injury. The helmet may comprise and/or may be configured to (e.g., detachably) accommodate the cranial cap.

The cranial cap and/or the helmet may comprise a connector port allowing fluidly coupling the pump with the cavity for controlling fluid pressure in the cavity based on a value relating to one or more of the sensed skin flap shape parameters.

In some examples, tubing comprising the connector port for connecting between the pump and the cavity may include a one-way valve. The one-way valve may be in a normally closed position, to seal the cavity unless the pump is connected with the cavity for reducing pressure in the cavity to cause, for example, lifting of the skin in distal direction to counteract forces that would, if the pressure in the cavity was not reduced by controllably engaging the pump, inadvertently cause excess sinking of the skin flap towards the brain tissue due to intracranial depressurization.

In some embodiments, the cranial cap may comprise a cap body having or defining a cap rim, and further include a flexible sheet (e.g., membrane) that is attached to the cap rim to form the pressure-controllable cavity. The sheet may be removably coupleable with (e.g., glued to) the skin flap. Once the cap is operably engaged with the patient's head, the sheet is overlaying the skin flap such that creating sub-pressure in the cavity causes lifting of the sheet in distal direction and, along with it, the lifting of the skin flap in distal direction. In some embodiments, the cranial cap and/or system may include the sheet and, optionally, the cap body, where the sheet and, optionally, the cap body include or are made of electroactive material.

In some other embodiments, the cap's rim may operably engage with the patient's head flap in a fluid-tight manner to form, with the patient's skin and cap body, a pressure-controllable cavity.

In some embodiments, the pump may be removably couplable with the cranial cap, as needed, for reducing the pressure in cavity to counteract sinking of the skin flap due to intracranial depressurization.

In some embodiments, the cap may be equipped or coupled with a cooling apparatus for cooling the temperature of a patient's skin and underlying tissue. In some examples, fluid in the cavity may be cooled to a desired low-temperature, e.g., by a heat exchanger or heat pump system.

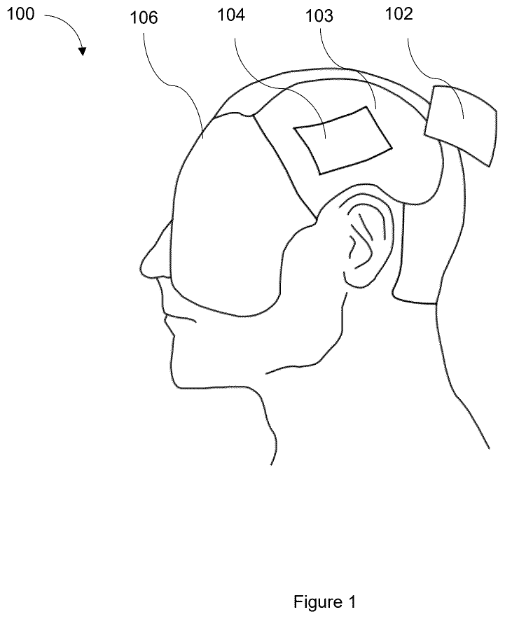

FIG. 1 schematically depicts a craniectomy region where a cranial bone portion 102 is removed leaving an aperture 104 in the remaining cranial bone 103 which is covered over by a skin flap 106. The aperture 104 may be larger or smaller and the skin flap 106 may be generally sufficiently large to cover the removed skull portion. The illustration is intended merely as an example and is not intended to be limiting in any way or even to accurately represent current protocols.

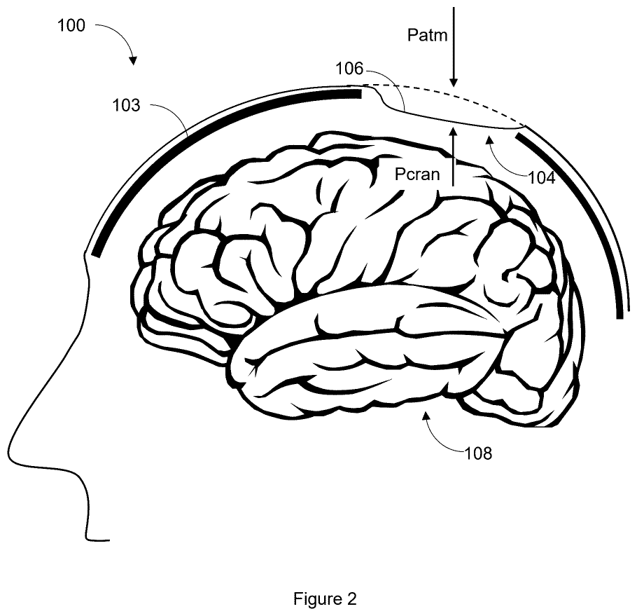

Further reference is made to FIG. 2. In some cases, the “sinking skin syndrome” may develop, which is the result of decreased intracranial pressure Pcran applied onto the inner skin surface of the skin flap 106 relative to atmospheric pressure Patm exerted onto the external skin surface of the skin flap 106, so that a pressure gradient Patm-Pcran=ΔP>0 across the skin flap 106 directly acts on the brain 108 resulting in brain tissue compression.

The present methods, devices and systems provide solutions for the changes in pressure due to position, movement and/or other factors. There is provided, for example, a cranial cap, adapted for covering at least a portion of a head having undergone a craniectomy.

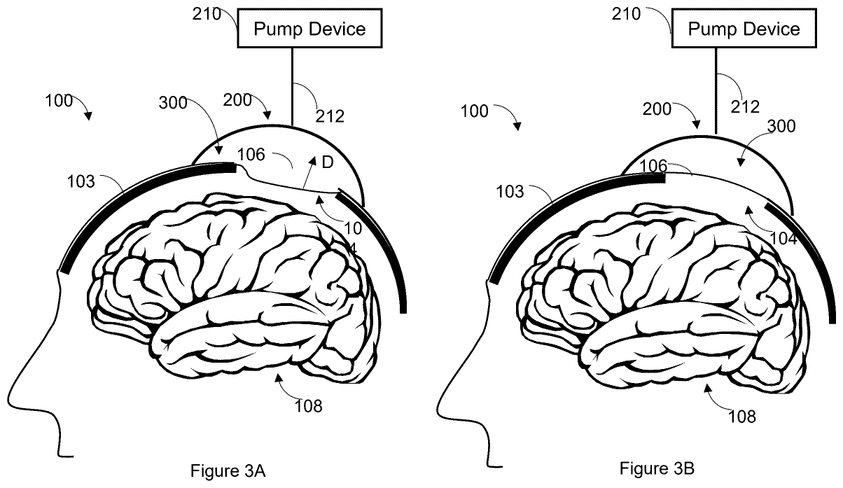

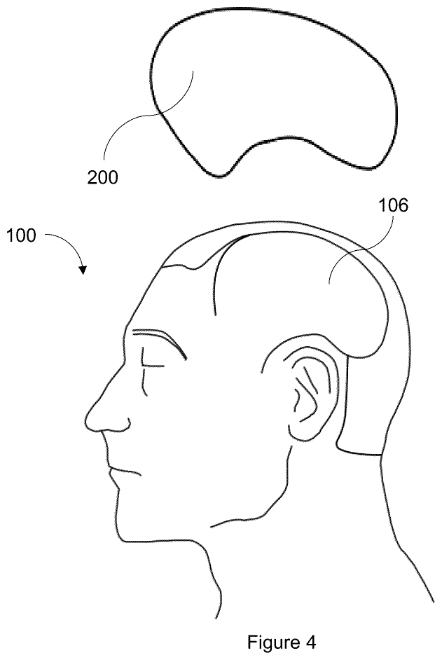

Additional reference is made to FIGS. 3A and 3B, which schematically depict a patient 100 having undergone a craniectomy, with the skin flap 106 returned to cover the aperture 104, and cranial cap 200 operably engaging with patient's head to cover aperture 104. FIG. 4 shows the cranial cap apart from the head of patient 100.

In some example implementations, cranial cap 200 may be custom sized and shaped to cover the surgical site, so that the edges of the cap 200 rest on skin that is supported by remaining cranial bone 103 surrounding the area of the removed cranial bone portion 102.

In some embodiments, the cap 200 may allow creating a fluid-tight cavity 300 above the aperture 104 covered by the skin flap 106. The cap 200 may be part of a system which is configured to allow sensing pressure imbalance between the intracranial compartment and the outer atmospheric pressure causing depression or other deformation of the skin flap 106, as shown in FIG. 3A. To correct for such pressure imbalance, negative pressure can be applied over the skin flap to lift it up, away from the brain 108, in distal direction D, to arrive at or maintain a desired skin flap configuration, which is schematically shown in FIG. 3B. The system may comprise a pump 210 that is removably couplable with the cap 200 in a manner to create a fluid path 212 between cavity 300 and a fluid source (not shown) and which is configured to apply suction or aspiration of fluid contained in cavity 300 to reduce the pressure therein and lift skin flap 106 in the distal direction D.

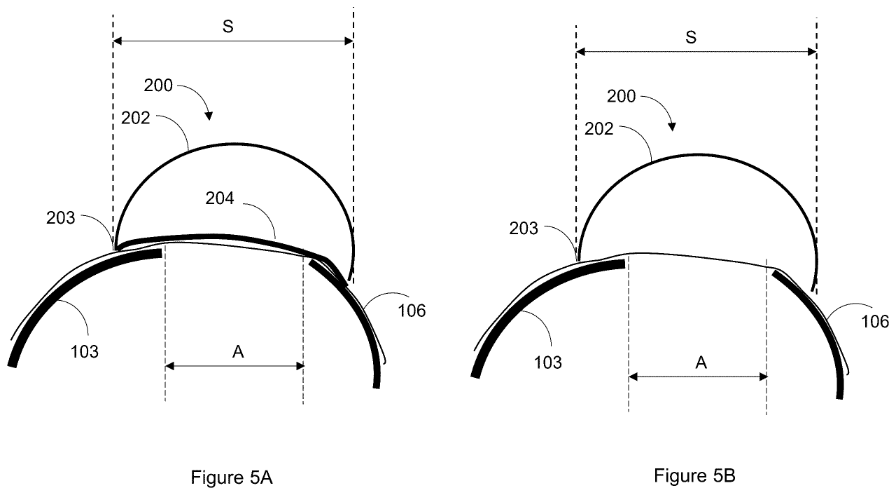

Cavity 300 may be formed in various ways. In the example schematically shown in FIG. 5A, cranial cap 200 may comprise a cap body 202 that may be made of stiff or rigid (also: substantially stiff or rigid), at least for the purposes of the applications described herein. Cap body 202 may have a concave shape (e.g., bulge outwards) with respect to the patient's skull when operably engaged therewith). Cap body 202 may define a cap rim 203.

In some examples, cap 200 may be made of transparent and lightweight material, having some degree of flexibility. Materials that can be used include, for example, silicon, polymer-based materials, and/or the like. Cap 200 may in some examples be custom-made for each patient, and/or pliable. In some examples, cap rim 203 may be made of rubber, intended to provide a seal to the cavity underneath the cap to allow control of the pressure in the cavity.

In some embodiments, cap 200 may additionally or alternatively include a flexible sheet 204. In some examples, cap 200 including sheet 204 may be devoid of cap body 202. In some embodiments, sheet 204 may be attached to cap rim 203 to form cavity 300, which may be a fluid-tight cavity. Sheet 204 may be flexible enough to be conformably engageable with the person's skull and, optionally, to entirely cover the underlying area of aperture 104. In some examples, geometry of sheet S may be such to cover any diameter of area A in excess overlap, e.g., as is schematically illustrated in FIG. 5A. Sheet 204 may be removably coupleable with (e.g., glued to) skin flap 106. Once cap 200 is operably engaged with the patient's head, sheet 204 is overlaying skin flap 106 such that creating sufficient sub-pressure in cavity 300 can cause lifting of the sheet in distal direction and, along with it, lift of skin flap 106 in distal direction. In some examples, sheet 204 may be preformed, flexible, and/or adapted to adhere to the patient's outer skin surface in a fluid tight (e.g., airtight) manner to allow creating a vacuum in cavity 300.

In some examples, a portion or the entirety of cap body 202 and/or of sheet 204 may include electroactive material capable of producing an output signal responsive to a change in shape of cap body 202 and/or of sheet 204. In some examples, a portion or the entirety of cap body 202 and/or of sheet 204 may include electroactive material capable of receiving a control signal causing a change in a shape and/or in any other characteristic and/or property of the electroactive material.

In the example shown in FIG. 5B, rim 203 may be configured to be directly attachable (e.g., glued) in a fluid tight manner onto the patient's outer skin surface such that the patient's cap body and the underlying skin portion form cavity 300, thus obviating the need of employing sheet 204.

In some embodiments, the shape of cap 200 may be determined by manually attained measurements and/or computer-aided scans of the operation site. Cap 200 may be custom made or come in prefabricated sizes and/or shapes that are not custom-made. Cap 200 may be formed using any manufacturing method including, but not limited to: 3D printing, injection molding and/or thermoforming.

Sheet 204 of cap 200 may be flexible, so as to deform (e.g., stretch) with natural movement of the head as well as expanding and contracting with the changes in pressure under the cap due to changes in intracranial pressure and/or atmospheric pressure due to the changes in position of the patient (e.g., when moving from a horizontal position to a vertical position) and/or pressure changes introduced into the space or cavity under the cap by pump 210 (see FIGS. 3A and 3B).

In operation, pump 210 creates a pressure imbalance causing the aspiration of fluid out of cavity 300, which in turn causes the skin flap 106 to be lifted in distal direction.

In order to facilitate such functionality, cavity 300 must be non-permeable to fluid. For example, sheet 204 and cap body 202 form a fluid tight seal. In another example, a layer of adhesive or semi-adhesive material may line rim 203 to afford the aforementioned functionality (air-tight seal) between the rim and skin.

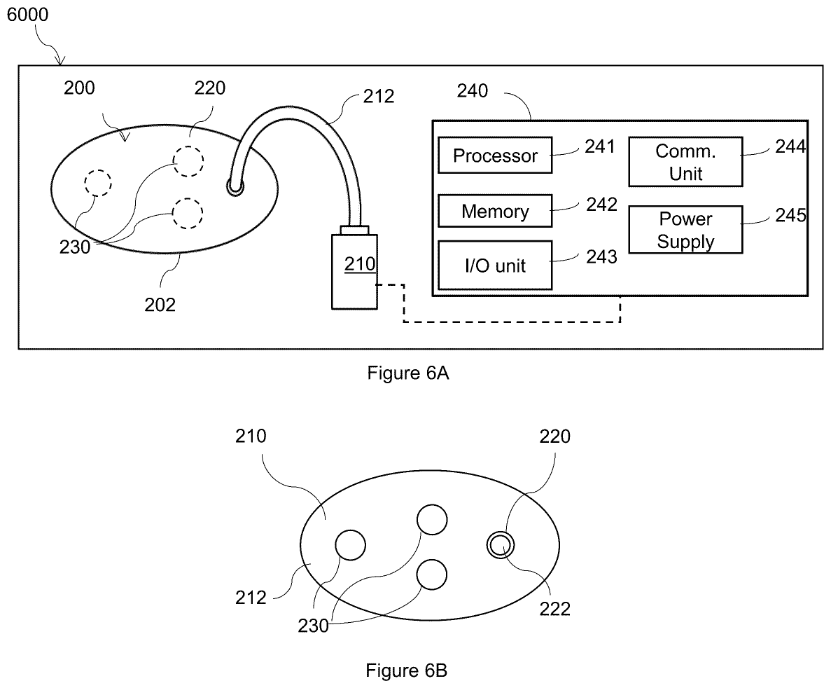

As shown in FIGS. 3A and 3B, and further in FIGS. 6A-6B, pump 210 regulates or modifies the fluid pressure between the cap 200 and site of interest on the head of the patient. The term “site of interest”, as used herein, may refer to the section of the head including the skin flap 106 that covers the removed cranial bone portion 102 as well as skin covering bone surrounding the aperture. The site of interest may include the entire skin flap together with additional skin surrounding the flap, a portion of the skin flap that covers at least the aperture as well as surrounding skull, or the precisely the skin flap, defined by the contours thereof.

Reference is now made to FIGS. 6A and 6B. FIG. 6A schematically illustrates a top view of an example embodiment of a SoT prevention system 6000 including a top view of cap 200, and FIG. 6B schematically illustrates an underside of cap 200. SoT Prevention System 6000 may include pump 210, tubing 212 for implementing a corresponding fluid path, a control unit 240, and one or more sensors 230. At least one sensor 230 may be arranged underneath and/or incorporated in cap 200, e.g., for monitoring skin flap shape parameters, patient movement and/or patient posture. In some embodiments, at least one sensor 230 may be employed remotely from cap 200, e.g., incorporated in a Smartwatch, or another wearable worn by the user, e.g., for monitoring patient movement and/or posture.

It is noted that while cap 200 is depicted as having a regular, oval shape, this should by no means be construed in a limiting manner, and that the depiction is merely an example and/or medium for conveying various components of the system, without being limiting in any way.

Pump 210, control unit 240 and sensors 230 are communicably coupled with each other (e.g., in a wired and/or wireless manner) such that control unit 240 can control pump operation based on skin flap shape parameters output values provided by sensors 230.

SoT Prevention System 6000 may further comprise a control unit 240 which may comprise a processor 241 and a memory 242. Memory 242 may be configured to store data and software code which, when executed by processor 241, results in a SoT prevention application. Such SoT prevention application may apply protocols for sensing changes in skin flap shape.

Memory 242 may further allow data storage of measurements and/or of action logs.

SoT prevention system 6000 may further include an I/O unit 243 for example, for displaying status data, possibly measurements results and possibly historic data logs; and a communication module 244 for allowing communication of data within the unit or with external systems.

An SoT Prevention System (e.g., system 6000) may further include a power supply for powering the various components of the systems disclosed herein. A power supply may be included in the cap and/or external to the cap. For example, power supply 245 may be incorporated in the cap and/or external to the cap.

In embodiments, the pump 210 is connected via a tubing 212 to the cap 200 in a permanent manner. Tubing 212 is connected to the cap via a fluid tight port 220 and/or a valve 222. When permanently connected to a pump, it may be expected that the pump continuously provides the requisite amount of positive or negative pressure, thereby obviating the need for a valve in the port 220. However, as a failsafe, the connector port 220 may (in embodiments) include valve 222 which seals the port in the absence of either negative or positive pressure from the pump.

In other embodiments, pump 210, (e.g., via tubing 212) may be detachable, adapted to be detached for periods of time (whether long or short), including extended periods of time, or removably couplable with the cranial cap 200 for bringing cavity 300 via the tubing, into fluid communication with pump 210.

There may be provided, according to some embodiments, a connector port 220 for removably coupling tubing 212 with cap body 202 for controlling or managing fluid pressure in cavity 300 formed, for example, between sheet 204 and cap body 202 in one embodiment, or directly between skin flap 106 and cap body 202, in another embodiment.

In some example configurations, connector port 220 may be coupled with tubing 212 and be adapted to couple with valve 222 in the cranial cap. According to a further option, the tubing 212 or a tubing portion may be adapted to couple with valve 222.

In embodiments, connector port 220 has a valve reversibly providing a fluid tight seal between the cap and the ambient air. The valve maintains the pressure level set by the pump (positive or negative) between the cap and the head, even after the tubing 212 and pump 210 have been disconnected. Such an arrangement allows the patient to be free of the pump (i.e., not having to be connected with the pump via the tubing), at least for a portion of time during the day.

The system or assembly further includes a control unit 240, wherein the control unit is configured to receive the sensor output produced by the at least one sensor 230 (see below) and to control, based on the received sensor output, the volume of the cavity. In some examples, a controller unit may be embodied as a controller or include a controller.

According to another configuration (not shown), pump 210 and control unit 240 are embedded in, or operationally coupled to, the cranial cap. According to this configuration, the miniaturized components work autonomously and/or under wireless control from a remote station.

In embodiments, pump 210 is controlled to reduce excess intracranial pressure.

FIG. 6A illustrates an internal view of an example embodiment of the cranial cap. An example valve 222 is visible in the internal view of the cranial cap 200. The example valve 222 is depicted as part of the connector port 220.

In some examples, valve 222 may be opened by coupling tubing 212 to the connector port 220 and/or by pressurized fluid conducted through the valve (when coupled to the tube) by pump 210, via tubing 212.

In some embodiments, valve 222 may be considered as having a normally-closed resting state, and/or can be defined as normally closed, and/or is closed in a resting state. The valve may be a one-way valve. In some examples, the one-way valve may be forced open when the tubing is coupled to the connector. In some embodiments, valve 222 may be a two-way valve that is able to withstand at least a predefined amount of pressure (i.e., that is within expected parameters) from ambient and/or intracranial sources. That is to say that normal or expected changes in pressure between the cap and head of the patient (also referred to herein as “under/below the cap”) and/or pressure exerted on the valve from outside the cap (also referred to herein as “above the cap”) will not open the valve. In order to open such a valve, in some examples, the pressure from the pump must exceed these parameters (at least initially).

Another functionality of the cranial cap is a sensing function. To facilitate one or more sensing functions, there is/are provided one or more sensors 230 on the internal or underside of cap body 202, which may herein also be referred to as “covering member”.

In embodiments, at least one sensor 230 is adapted to be disposed between sheet 204 and the head/site of interest to sense a magnitude of one or more physical characteristics relating to the cavity and/or the skin flap and/or at least one intracranial physiological state such as intracranial pressure. The one or more physical characteristics may include, for example, fluid pressure in the cavity; a cavity height between the cranial cap and flexible membrane; (e.g., skin) pulsativity; temperature in the cavity; skin flap temperature; or any combination of the aforesaid. In some embodiments, the magnitude of at least one more physiological characteristic may be sensed for deriving information about the intracranial physiological state of a patient such as intracranial pressure.

In embodiments where the cap does not employ sheet 204, at least one sensor 230 is adapted to be disposed between the site of interest to directly face the skin flap for sensing one or more physical characteristics of cavity 300.

Sensors 230 can be any one or more of the following: a pressure sensor, a barometric pressure sensor, an imaging sensor/camera, a distance measurement sensor, a temperature sensor, inertial sensors (e.g., gyroscope, and an accelerometer).

In some examples, outputs provided by a plurality of the sensors may be compared with each other to determine a level a correspondence between the provided outputs, for instance, to reduce the probability of a “false-positive” output, indicating that the skin flap has sunken to an extent requiring the lifting of the skin flap.

Sensors 230 may be active for monitoring skin flap 106 relating parameters when pump 210 is connected, when pump 210 is disconnected, or at both times.

A pressure sensor may be used, for example, to sense the pressure within cavity 300. An excess increase in the sensed pressure may indicate, for example, that a desired level of cavity pressure is not being maintained and that the “sinking skin” phenomenon may occur.

An imaging sensor may be employed for imaging the site of interest for various monitoring reasons, such as, for example, shape of the skin flap (e.g., to detect if the flap is curving or sinking into the cranial cavity, detection of inflammation at the site of interest (that may be indicative of an infection or other ailment), and/or abnormal growths and/or skin conditions developing under the cap (e.g., if fitted for an extended period of time).

The sensors may indicate that some failing of the (fluid pressure) system resulting in the loss of desired fluid pressure. Alternatively, or additionally, the sensors may indicate swelling of the area.

A temperature sensor may be employed to monitor the internal temperature under the cap for sensing clinical parameter values and/or determining the patient's level of comfort.

Inertial sensors may be employed to monitor the motion of the patient. As mentioned elsewhere herein, change in orientation (e.g., from horizontal to vertical) may change the intracranial pressure. In a similar fashion, the inertial sensors may be employed to monitor movement of the patient that may momentarily affect the pressure levels under the cap.

Sensors 230 may also be employed for determining material integrity of cap 200, e.g., to detect leakage of fluid from cavity 300.

The cranial cap 200 further includes wired or wireless communications components for the transmission of electronic signals output by the at least one sensor.

In some embodiments, sensors 230 only record and transmit data/electronic signals when connected with control unit 240. In some embodiments, sensors 230 constantly monitor and store the sensor data. For example, the sensors may have a local resource (e.g., a System on a Chip, SoC) on or in cap 200 for powering the sensors and/or storing continuous data. When connected with the control unit, the stored sensor data is transferred to control unit 240 for processing and analysis. In some embodiments, a local power source (e.g., part of the SoC) may be recharged each time the cap is connected with the pump 210 and/or control unit 240.

In some examples, sensors 230 may be activated at predetermined time stamps (e.g., every few minutes, or hours). In some examples, sensors activation time may be adaptively set, for example, based on a previously sensed physical characteristic relating to the patient. For example, increased patient activity sensed by inertial sensors may prompt that for example a pressure sensor configured for sensing cavity pressure is activated more often, over some period of time.

In some embodiments, sensors 230 may be powered by power supply 245 of control unit 240.

Alternatively, or additionally, the sensors may be wirelessly coupled to the control unit. Many variations and configurations can be employed. One example is that the sensors are coupled (e.g., in a wired manner) to a wireless communications component (not shown) which transmits the sensor data (continuously or at predetermined intervals) directly or indirectly (e.g., via a personal mobile device) to control unit 240.

The following exemplifies an operating protocol of SoT Prevention system 6000.

In some examples, a preset parameter set is input into the memory 242. This parameter set can be according to known practices, standard operating procedures, base line measurement done during the installation and placement of the cap or communicated from the physician periodically based on periodic evaluation. the parameters may depend on the injury type and craniectomy procedure, the extent of the craniectomy, and may include several sets of desired parameters depending on conditions such as body position, such lying down at night, walking and standing or sitting for work.

In some examples, sensors 230 may be controlled to sense physical characteristics periodically or continuously. Sensor outputs descriptive of (also: relating to) skin flap shape parameter values are received by memory 242, which are then compared with preset skin flap shape parameter values and/or to previously sensed skin flap shape parameter values to detect changes that can imply either cap function conditions, such a fluid leakage, change to physiological conditions of the tissue under the skin flap, such an inflammation.

If a change is detected between current parameters and the preset or previous parameters that meets a “skin-flap depression criterion”, SoT Prevention System 6000 provides a corresponding output, for example, to adjust cavity pressure automatically and/or alert a user about the need to connect pump 210 with cap 200. Generally, the skin-flap criterion may be defined by a threshold defining what may be considered that the skin flap is (excessively) sunken. This depression-threshold may be a threshold which is static, dynamic or adaptive. Static thresholds are predetermined thresholds that remain constant. Dynamic thresholds are forcefully changed, for example, at a certain time of day, or a certain day of the year. Adaptive thresholds are changed in response to changes, for example, in patient characteristics and may vary, depending on a variety of parameters.

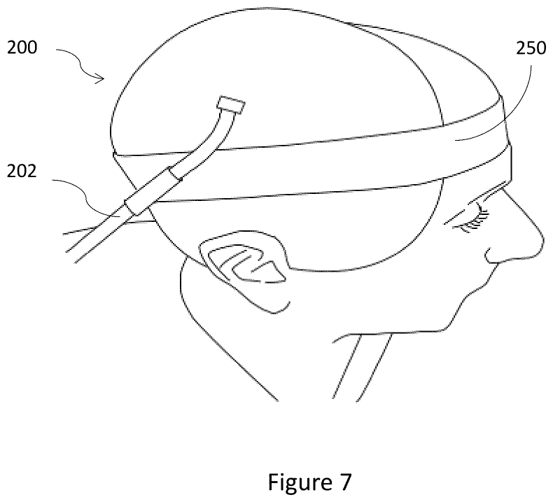

Additional reference is now made to FIG. 7, which schematically depicts a patient with the cranial cap of the present disclosure, including, for example, a coupling member 250 (e.g., a band) coupled to the covering member/piece of sheet 204 for coupling the cranial cap 200 to the head.

It is noted that, in some embodiments, a cranial cap may be held in operable position with a patient's head solely through negative pressure suction applied and/or maintained between the underside of the cranial cap and the external side of the patient's skin.

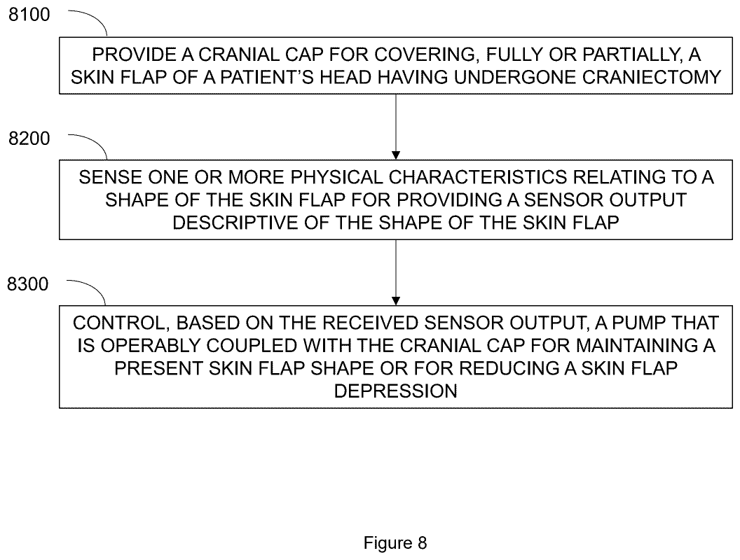

Further reference is made to FIG. 8. According to some embodiments, a method for treating or preventing SOT may comprise, for example, providing a cranial cap for covering, fully or partially, a skin flap of a patient's head having undergone craniectomy (block 8100).

In some embodiments, the method may include sensing one or more physical characteristics relating to a shape of the skin flap for providing a sensor output descriptive of the shape of the skin flap (8200).

In some embodiments, the method may further include controlling, based on the received sensor output, a pump that is operably coupled with the cranial cap for maintaining a present skin flap shape or for reducing a skin flap depression (block 8300).

### ADDITIONAL EXAMPLES

Example 1 concerns a cranial cap, adapted for covering, at least partially or fully, a skin flap of a patient's head having undergone a craniectomy, the cranial cap comprising:
a cap body;
a flexible sheet adaptable to adhere to the head;
wherein the cap body and the flexible sheet form a fluid-tight cavity;
a tubing connector port for removably bringing a pump in fluid communication with the cavity such to enable controlling fluid pressure in the cavity; and
at least one sensor adapted and disposed to sense a physical characteristic relating to a skin flap shape for responsively providing an output descriptive of the skin flap shape.

Example 2 includes the subject matter of Example 1 and, optionally, wherein the cranial cap is custom manufactured to contours of the portion of the head being covered.

Example 3 includes the subject matter of examples 1 and/or 2 and, optionally, wherein the cranial cap is manufactured using one of the following methods: 3D printing, injection molding, thermoforming, or any combination of the aforesaid.

Example 4 includes the subject matter of any one or more of the examples 1 to 3 and, optionally, wherein the one or more sensors include one of the following: a pressure sensor, a barometric pressure sensor, an imaging, a proximity sensor, a temperature sensor, a gyroscope, an accelerometer, or any combination of the aforesaid.

Example 5 includes the subject matter of any one or more of the examples 1 to 4 and, optionally, wherein the at least one sensor is or can be operably coupled with the pump and a control unit.

Example 6 includes the subject matter of any one or more of the examples 1 to 5 and, optionally, wherein the cranial cap further includes wired and/or wireless communication components for the transmission of electronic signals that are output by the at least one sensor to a control unit.

Example 7 concerns a cranial cap adapted for covering, at least partially or fully, a skin flap of a patient's head having undergone a craniectomy, the cranial cap comprising:
a cap body;
wherein the cap body defines a rim that is configured for engaging with the patient's head to create a fluid-tight cavity that is confined by the cap body and the underlining skin flap;
a tubing connector port for removably bringing a pump in fluid communication with the cavity such to enable controlling fluid pressure in the cavity; and
at least one sensor adapted and disposed to sense a physical characteristic relating to a skin flap shape for responsively providing an output descriptive of the skin flap shape.

Example 8 includes the subject matter of example 7 and, optionally, wherein the cranial cap is custom manufactured to contours of the portion of the head being covered.

Example 9 includes the subject matter of examples 7 and/or 8 and, optionally, wherein the cranial cap is manufactured by a method selected from the group including: 3D printing, injection molding and thermoforming.

Example 10 includes the subject matter of any one or more of the examples 7 to 9 and, optionally, wherein the one or more sensors include one of the following: a pressure sensor, a barometric pressure sensor, an imaging, a proximity sensor, a temperature sensor, a gyroscope, an accelerometer, or any combination of the aforesaid.

Example 11 includes the subject matter of any one or more of the examples 7 to 10 and, optionally, wherein the at least one sensor and the pump are in communication with a control unit.

Example 12 includes the subject matter of any one or more of the examples 7 to 11 and, optionally, wherein the cranial cap further includes wired and/or wireless communication components for the transmission of electronic signals that are output by the at least one sensor to a control unit.

Example 13 pertains to a system for preventing Syndrome of the Trephined (SoT), comprising:
the cranial cap of any one of the examples 1 to 12, and
a control unit that is operably coupled with the at least one sensor and the pump, wherein the at least one sensor adapted and disposed to sense one or more physical characteristics relating to a shape of the skin flap for responsively providing an output descriptive of the skin flap shape;
wherein the control unit is configured to receive the sensor outputs for controlling, based on the received sensor outputs, the pump to achieve one or more of the following: maintain a present skin flap shape; and reduce or eliminate a skin flap depression.

Example 14 includes the subject matter of any one or more of the examples 1 to 13 and, optionally, wherein the pump is configured to controllably aspirate fluid from the cavity to lift a sunken skin flap towards the cap to a desired height.

Example 15 includes the subject matter of any one or more of the examples 13 and/or 14, wherein the pump is controlled to reduce excess intracranial pressure.

Example 16 includes the subject matter of any one or more of the examples 13 to 15 and, optionally, wherein the at least one physical characteristic relating to the cavity sensed by the at least one sensor includes one or more of the following:
fluid pressure in the cavity, a cavity height between the cap body and the flexible membrane, cavity height between the cap body and the skin flap, a cavity volume, or any combination of the aforesaid.

Example 17 includes the subject matter of any one or more of the examples 13 to 16 and, optionally at least one valve for controlling the flow of fluid between a fluid source and the cavity via the pump.

Example 18 pertains to a method for preventing SoT, the method comprising:
providing a cranial cap for covering, fully or partially, a skin flap of a patient's head having undergone craniectomy;
sensing one or more physical characteristics relating to a shape of the skin flap for providing a sensor output descriptive of the shape of the skin flap; and
controlling, based on the received sensor output, a pump that is operably coupled with the cranial cap for maintaining a present skin flap shape or for reducing a skin flap depression.

Example 19 includes the subject matter of example 18 and, optionally, a cranial cap according to any one or more of the examples 1 to 12.

Example 20 includes the subject matter of examples 18 and/or 19, performed by employing a system according to any one or more of the examples 13 to 17.

An example includes a helmet that can be worn by the patient to protect its head, wherein the helmet comprises and/or is configured to detachably accommodate the cranial cap according to any one of the example and/or embodiments described herein.

Embodiments pertain to a cranial cap, adapted for covering, at least partially or fully, a skin flap of a patient's head having undergone a craniectomy, the cranial cap comprising:
a cap body;
a flexible sheet adaptable to adhere to the head, wherein the cap body and the flexible sheet are sealed to form a fluid-tight cavity;
a tubing connector port for removably bringing a pump in fluid communication with the cavity such to enable controlling fluid pressure in the cavity; and
at least one wearable sensor adapted and disposed to sense at least one characteristic relating to
a) a skin flap shape; and
b) patient posture and/or patient movement
for providing at least one sensor output descriptive of or relating to the at least one sensed characteristic.

In some embodiments, the at least one sensor output pertains for example to one or more of the following: magnitude of skin flap depression; skin flap sinking or rise rate; posture transitions sequence; frequency of posture transitions; type of a current posture; and/or physical activity of the patient.

In some embodiments, the cranial cap is manufactured by employing, for example, 3D printing, injection molding, thermoforming or any combination of the aforesaid.

In some embodiments, wherein the at least one sensor is configured to implement one of the following: a pressure sensor, a barometric pressure sensor, an imaging, a proximity sensor, a temperature sensor, a gyroscope, an accelerometer, or any combination of the aforesaid.

In some embodiments, the at least one sensor is or can be operably coupled with the pump and a control unit.

In some embodiments, wherein the cranial cap further includes wired and/or wireless communication components for the transmission of electronic signals that are output by the at least one sensor to a control unit.

In some embodiments, the at least one characteristic relating to the skin flap shape includes one of the following: fluid pressure in the cavity; a cavity height between the cranial cap and flexible membrane; pulsativity; intracranial pressure, temperature in the cavity; temperature of the skin flap; or any combination of the aforesaid.

Embodiments include a helmet that can be worn by the patient, wherein the helmet comprises and/or is configured to detachably accommodate the cranial cap.

In some embodiments, a cranial cap is provided adapted for covering, at least partially or fully, a skin flap of a patient's head having undergone a craniectomy, the cranial cap comprising:
a cap body;
wherein the cap body defines a rim that is configured for sealingly engaging with the patient's head to create a fluid-tight cavity that is confined by the cap body and the underlining skin flap;
a tubing connector port for removably bringing a pump in fluid communication with the cavity such to enable controlling fluid pressure in the cavity; and at least one sensor adapted and disposed to sense: at least one characteristic relating to a skin flap shape; and patient posture and/or patient movement, for providing at least one sensor output descriptive of or relating to the at least one characteristic.

In some embodiments, the cranial cap is custom manufactured to contours of a portion of the head being covered.

Embodiments pertain to a system for preventing Syndrome of the Trephined (SoT) for a subject, the system comprising: at least one sensor wearable by the subject, wherein the at least one sensor is adapted and disposed to sense at least one characteristic relating to: a skin flap shape; and patient posture and/or patient movement, for providing at least one sensor output descriptive of or relating to the at least one characteristic; a cranial cap comprising; a flexible sheet adaptable to adhere to the head; wherein the cap body and the flexible sheet are sealed to form a fluid-tight cavity; a tubing connector port for removably bringing a pump in fluid communication with the cavity such to enable controlling fluid pressure in the cavity, a control unit that is operably coupled with the at least one sensor and the pump, and configured to receive the at least one sensor output for controlling, based on the received at least one sensor output, the pump to achieve one or more of the following: maintain a present skin flap shape; and reduce or eliminate a skin flap depression, for different patient postures and/or during patient movement.

In some embodiments, the pump is configured to controllably aspirate fluid from the cavity to lift a sunken skin flap towards the cap to a desired height.

In some embodiments, the pump is controlled to reduce excess intracranial pressure.

In some embodiments, the at least one physical characteristic relating to the cavity sensed by the at least one sensor includes one or more of the following: fluid pressure in the cavity, a cavity height between the cap body and the flexible membrane, cavity height between the cap body and the skin flap, a cavity volume, or any combination of the aforesaid.

In some embodiments, the system further comprises at least one valve for controlling the flow of fluid between a fluid source and the cavity via the pump.

Embodiments pertain to a system for preventing Syndrome of the Trephined (SoT), comprising:
at least one wearable sensor adapted and disposed to sense at least one characteristic relating to a skin flap shape; and patient posture and/or patient movement for providing at least one sensor output descriptive of or relating to the at least one sensed characteristic;
a cap body, wherein the cap body defines a rim that is configured for sealingly engaging with the patient's head to create a fluid-tight cavity that is confined by the cap body and the underlining skin flap;
a tubing connector port for removably bringing a pump in fluid communication with the cavity such to enable controlling fluid pressure in the cavity;
a control unit that is operably coupled with the at least one sensor and the pump, and configured to receive the sensor outputs for controlling, based on the received sensor outputs, the pump to achieve one or more of the following: maintain a present skin flap shape; and reduce or eliminate a skin flap depression, for or at different patient postures and/or during patient movement.

The various features and steps discussed above, as well as other known equivalents for each such feature or step, can be mixed and matched by one of ordinary skills in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

Any digital computer system, module and/or engine exemplified herein can be configured or otherwise programmed to implement a method disclosed herein, and to the extent that the system, module and/or engine is configured to implement such a method, it is within the scope and spirit of the disclosure. Once the system, module and/or engine are programmed to perform particular functions pursuant to computer readable and executable instructions from program software that implements a method disclosed herein, it in effect becomes a special purpose computer particular to embodiments of the method disclosed herein. The methods and/or processes disclosed herein may be implemented as a computer program product that may be tangibly embodied in an information carrier including, for example, in a non-transitory tangible computer-readable and/or non-transitory tangible machine-readable storage device. The computer program product may be directly loadable into an internal memory of a digital computer, comprising software code portions for performing the methods and/or processes as disclosed herein. The term “non-transitory” is used to exclude transitory, propagating signals, but to otherwise include any volatile or non-volatile computer memory technology suitable to the application.

Additionally or alternatively, the methods and/or processes disclosed herein may be implemented as a computer program that may be intangibly embodied by a computer readable signal medium. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a non-transitory computer or machine-readable storage device and that can communicate, propagate, or transport a program for use by or in connection with apparatuses, systems, platforms, methods, operations and/or processes discussed herein.

The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by one or more communication networks.

These computer readable and executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

Unless otherwise specified, the terms ‘about’ and/or ‘close’ with respect to a magnitude or a numerical value may imply to be within an inclusive range of −10% to +10% of the respective magnitude or value.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

It should be understood that where the claims or specification refer to “a” or “an” element and/or feature, such reference is not to be construed as there being only one of that element. Hence, reference to “an element” or “at least one element” for instance may also encompass “one or more elements”.

As used herein the term “configuring” and/or ‘adapting’ for an objective, or a variation thereof, implies using materials and/or components in a manner designed for and/or implemented and/or operable or operative to achieve the objective.

Unless otherwise stated or applicable, the use of the expression “and/or” between the last two members of a list of options for selection indicates that a selection of one or more of the listed options is appropriate and may be made, and may be used interchangeably with the expressions “at least one of the following”, “any one of the following” or “one or more of the following”, followed by a listing of the various options.

As used herein, the phrase “A,B,C, or any combination of the aforesaid” should be interpreted as meaning all of the following: (i) A or B or C or any combination of A, B, and C, (ii) at least one of A, B, and C; and (iii) A, and/or B and/or C. This concept is illustrated for three elements (i.e., A,B,C), but extends to fewer and greater numbers of elements (e.g., A, B, C, D, etc.).

It is noted that the terms “operable to” or “operative to” can encompass the meaning of the term “adapted or configured to”. In other words, a machine “operable to” or “operative to” perform a task can in some embodiments, embrace a mere capability (e.g., “adapted”) to perform the function and, in some other embodiments, a machine that is actually made (e.g., “configured”) to perform the function.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 4, from 1 to 4, from 1 to 5, from 2 to 4, from 2 to 6, from 4 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It should be appreciated that combinations of features disclosed in different embodiments are also included within the scope of the present inventions.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A system for preventing Syndrome of the Trephined (SoT) in a patient, the system comprising:
at least one sensor wearable by the subject, wherein the at least one sensor is adapted and disposed to sense at least one characteristic relating to:
a) a skin flap shape; and
b) patient posture and/or patient movement
for providing at least one sensor output descriptive of or relating to the at least one characteristic;
a cranial cap comprising;
a flexible sheet adaptable to adhere to the head;
wherein the cap body and the flexible sheet are sealed to form a fluid-tight cavity;
a tubing connector port for removably bringing a pump in fluid communication with the cavity such to enable controlling fluid pressure in the cavity;
a control unit that is operably coupled with the at least one sensor and the pump, and configured to receive the at least one sensor output for controlling, based on the received at least one sensor output comprising the patient posture and/or movement, the pump to achieve one or more of the following:
maintain a present skin flap shape; and
reduce or eliminate a skin flap depression,
for or at different patient postures and/or during patient movement.

2. The SoT prevention system of claim 1, wherein the pump is configured to controllably aspirate fluid from the cavity to lift a sunken skin flap towards the cap to a desired height.

3. The SoT prevention system of claim 1, wherein the pump is controlled to reduce excess intracranial pressure.

4. The SoT prevention system of claim 1, wherein the at least one physical characteristic relating to the cavity sensed by the at least one sensor includes one or more of the following:
fluid pressure in the cavity, a cavity height between the cap body and the flexible membrane, cavity height between the cap body and the skin flap, a cavity volume, or any combination of the aforesaid.

5. The SoT prevention system of claim 1, further comprising at least one valve for controlling the flow of fluid between a fluid source and the cavity via the pump.

6. An SoT prevention system, comprising:
a cap body,
wherein the cap body includes electroactive material for sensing a physical magnitude relating to a shape of a skin flap;
at least one wearable inertial sensor adapted and disposed to sense at least one characteristic relating to patient posture and/or patient movement and to output a signal relating to the sensed patient posture and/or movement;
a pump that is in fluid communication with a cavity formed by the cap body and overlaying the skin flap;
at least one memory; and
at least one processor configured to execute instructions stored in the at least one memory for controlling, based on outputs provided by the electroactive material and the at least one wearable sensor, the pump for preventing or reducing SOT.

7. (canceled)

8. (canceled)

9. A cranial cap, adapted for covering, at least partially or fully, a skin flap of a patient's head having undergone a craniectomy, the cranial cap comprising:
a cap body;
a flexible sheet adaptable to adhere to the head;
wherein the cap body and the flexible sheet are sealed to form a fluid-tight cavity;
a tubing connector port for removably bringing a pump in fluid communication with the cavity such to enable controlling fluid pressure in the cavity; and
at least one wearable sensor adapted and disposed to sense at least one characteristic relating to
a) a skin flap shape; and
b) patient posture and/or patient movement
for providing at least one sensor output descriptive of or relating to the at least one sensed characteristic.

10. The cranial cap of claim 9, wherein the at least one sensor output pertains to one of the following:
magnitude of skin flap depression;
skin flap sinking rate;
posture transitions sequence;
frequency of posture transitions;
type of a current posture;
physical activity of the patient; or any combination of the aforesaid.

11. The cranial cap of claim 9, wherein the cranial cap is manufactured by employing: 3D printing, injection molding, thermoforming or any combination of the aforesaid.

12. The cranial cap of claim 9, wherein the at least one sensor is configured to implement one of the following: a sensor based on electroactive material, a pressure sensor, a barometric pressure sensor, an imaging sensor, a proximity sensor, a temperature sensor, a gyroscope, an accelerometer, or any combination of the aforesaid.

13. The cranial cap of claim 9, wherein the at least one sensor is or can be operably coupled with the pump, a control unit and/or electroactive material.

14. The cranial cap of claim 9, wherein the cranial cap further includes wired and/or wireless communication components for the transmission of electronic signals that are output by the at least one sensor to a control unit.

15. The cranial cap of claim 9, wherein the at least one characteristic relating to the skin flap shape includes one of the following:
fluid pressure in the cavity; a cavity height between the cranial cap and flexible membrane; pulsativity; intracranial pressure, temperature in the cavity; temperature of the skin flap; or any combination of the aforesaid.

16. The cranial cap of claim 9, wherein at least a portion of the cranial cap body includes electroactive material for sensing flap height and/or controlling flap height.

17. (canceled)

18. A cranial cap, adapted for covering, at least partially or fully, a skin flap of a patient's head having undergone a craniectomy, the cranial cap comprising:
a cap body;
wherein the cap body defines a rim that is configured for sealingly engaging with the patient's head to create a fluid-tight cavity that is confined by the cap body and the underlining skin flap;
a tubing connector port for removably bringing a pump in fluid communication with the cavity such to enable controlling fluid pressure in the cavity; and
at least one sensor adapted and disposed to sense:
a) at least one characteristic relating to a skin flap shape; and
b) patient posture and/or patient movement
for providing at least one sensor output descriptive of or relating to the at least one characteristic.

19. The cranial cap of claim 18, wherein the one or more sensors include one of the following: electroactive material, a pressure sensor, a barometric pressure sensor, an imaging, a proximity sensor, a temperature sensor, a gyroscope, an accelerometer, or any combination of the aforesaid.

20. The cranial cap of claim 18, wherein the at least one sensor and the pump are in communication with a control unit.

21. The cranial cap of claim 18, wherein the cranial cap further includes wired and/or wireless communication components for the transmission of electronic signals that are output by the at least one sensor to a control unit.

22. The cranial cap of claim 18, wherein the at least one characteristic relating to the skin flap shape includes one of the following:
fluid pressure in the cavity; a cavity height between the cranial cap and flexible membrane; pulsativity; intracranial pressure, temperature in the cavity; temperature of the skin flap; or any combination of the aforesaid.

23. (canceled)

24. (canceled)

25. The system of claim 1, further comprising a helmet that can be worn by the patient, the helmet being to configured to detachably accommodate a cranial cap.
